(19) United States
(12) Patent Application Publication
(10) Pub. No.: US 2022/0028501 A1
(43) Pub. Date: Jan. 27, 2022

(54) MEDICAL SUPPORT SYSTEM, MEDICAL SUPPORT DEVICE, AND MEDICAL SUPPORT METHOD

(71) Applicant: SONY GROUP CORPORATION, TOKYO (JP)

(72) Inventor: SUGURU DOWAKI, TOKYO (JP)

(21) Appl. No.: 17/296,713

(22) PCT Filed: Nov. 1, 2019
(86) PCT No.: PCT/JP2019/042964

§ 371 Date: May 25, 2021

(51) Int. Cl. G16H 10/40; G16H 70/60; G16H 30/00

(52) U.S. Cl. CPC G16H 10/40; G16H 30/00; G16H 70/60

(57) ABSTRACT

A medical support system (1000) according to an embodiment includes: a scanner (110) configured to scan an evaluation piece into which a specimen is processed and acquire an evaluation piece image; a server (120) configured to compare specimen information on the specimen and evaluation piece information that is obtained from the evaluation piece image and output a comparison result; and a display device (130) configured to present the comparison result to a user is provided. This makes it possible to reduce work of an operator.

### FIELD

The present disclosure relates to a medical support system, a medical support device, and a medical support method.

### BACKGROUND

In a medical setting, in order to consolidate information on patients, a management method of storing examination requests and examination data on specimens of patients in a hospital, examination requests and examination data that are based on patients outside the hospital and that are submitted from the outside of the hospital, etc., in association with one another is used.

For example, in Patent Literature 1, a hospital information system that consolidates patient information in a hospital, using identifying codes in an automatic lean method in anatomical pathology, is described. The technique described in Patent Literature 1 makes it possible to identify and manage orders of biopsies in the hospital information system, cassettes that are used for paraffin blocks, and pathological slides.

### CITATION LIST

### Patent Literature

Patent Literature 1: Japanese National Publication of International Patent Application No. 2009-520186

### SUMMARY

### Technical Problem

The technique described in Patent Literature 1, however, requires, at the stage where an operator processes a specimen that is sampled from a living body, checking information on the specimen in each time, which increases work of the operator. In consideration of the above-described circumstances, it is preferable to reduce the work of the operator.

### Solution to Problem

For solving the problem described above, a medical support system according to one aspect of the present disclosure has a scanner configured to scan an evaluation piece into which a specimen is processed and acquire an evaluation piece image; a server configured to compare specimen information on the specimen and evaluation piece information that is obtained from the evaluation piece image and output a comparison result; and a display device configured to present the comparison result to a user.

For solving the problem described above, a medical support device according to one aspect of the present disclosure has an acquisition unit configured to scan an evaluation piece into which a specimen is processed and acquire an evaluation piece image; a comparison result output unit configured to compare specimen information on the specimen and evaluation piece information that is obtained from the evaluation piece image and output a comparison result; and a display unit configured to present the comparison result to a user.

A medical support method according to one aspect of the present disclosure comprises, by a scanner, scanning an evaluation piece into which a specimen is processed and acquiring an evaluation piece image; by a calculation device, comparing specimen information on the specimen and evaluation piece information that is obtained from the evaluation piece image and outputting a comparison result; and by a display device, presenting the comparison result to a user.

### BRIEF DESCRIPTION OF DRAWINGS

FIG. 1 is an overview diagram illustrating an overview according to an embodiment of the disclosure.

FIG. 2 is a diagram illustrating a general flow of pathological diagnosis.

FIG. 3 is a block diagram illustrating a configuration of a medical support system according to the embodiment of the disclosure.

FIG. 4 is a schematic diagram illustrating an example of operations of the medical support system according to the embodiment.

FIG. 5 is a schematic diagram illustrating an example of operations of the medical support system according to the embodiment.

FIG. 6 is a schematic diagram illustrating an example of operations of the medical support system according to the embodiment.

FIG. 7 is a diagram illustrating an example of display on a display device of the medical support system according to the embodiment.

FIG. 8 is a diagram illustrating an example of display on the display device of the medical support system according to the embodiment.

FIG. 9 is a diagram illustrating an example display on the display device of the medical support system according to the embodiment.

FIG. 10 is a flowchart illustrating an example of a flow of operations of the medical support system according to the embodiment.

FIG. 11 is a diagram illustrating an example of modification of the medical support system according to the embodiment.

FIG. 12 is a diagram illustrating an example of modification of the medical support system according to the embodiment.

FIG. 13 is a diagram illustrating an example of modification of the medical support system according to the embodiment.

FIG. 14 is a diagram illustrating an example of modification of the medical support system according to the embodiment.

FIG. 15 is a diagram illustrating an example of a hardware configuration of the medical support system according to the embodiment.

### DESCRIPTION OF EMBODIMENTS

Preferable embodiments of the disclosure will be described in detail below with reference to the accompanying drawings. In the specification and the drawings, components having functional configurations that are substantially the same are denoted with the same reference number and thus redundant description thereof is omitted.

Description will be given in the following order.

1. Overview of Technique

2. Embodiment
2.1. Configuration
2.2. Flow of Operations

3. Modification

4. Hardware Configuration

### 1. Overview of Technique

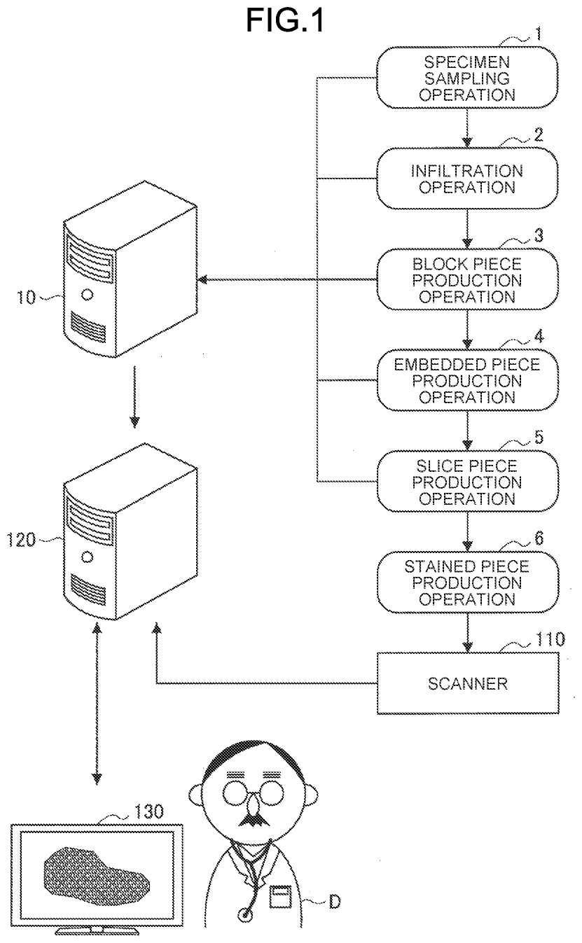

With reference to FIG. 1, the technical overview of a medical support system 1000 that can reduce work of an operator will be described. FIG. 1 is a diagram to describe the overview of the medical support system 1000. For example, pathology diagnosis is taken as an example where steps taken by an operator are divided into an operation 1 of sampling a specimen from a living body; an infiltration operation 2 of infiltrating the specimen, which is sampled from the living body, with an organic solvent; a block piece production operation 3 of producing a block piece by cutting the specimen; an embedded piece production operation 4 of fixing the block piece with paraffin, or the like, and producing an embedded piece; and a slice piece production operation 5 of producing a slice piece by slicing the fixed block piece; and a stained piece production operation 6 of staining the slice piece to produce a stained piece.

In each of the operations 1 to 6, the operator inputs specimen information on a specimen that is processed, containing the orientation of the specimen with respect to a pathology slide, to a hospital information system 10 and checks the specimen information per operation.

As described above, when performing each of the operations 1 to 6, the operator has much work including, per operation, checking the specimen information before and after the operation and further checking specimen information to be input. There is a possibility that specimens may be mixed up in each operation due to manual operation by the operator. In consideration of the circumstances, reducing the operational work has been required.

The medical support system 1000 captures and acquires an image of the stained piece after the stained piece production operation 6, using a scanner 110, thereby acquiring an evaluation piece to be used for pathology diagnosis.

The medical support system 1000 determines whether there is no mixing up or whether the evaluation piece and the specimen agree in each operation by comparing the acquired evaluation piece and the specimen information in each of the operations 1 to 6, using a server 120, and outputs a comparison result, using a display device 130. Accordingly, it is possible to reduce the operation work of the operator or promptly determine occurrence of human error in specimen processing. When an agreement between the specimen and the evaluation piece is confirmed from the output of the comparison result, for example, a pathologist D checks by sight the evaluation piece that is displayed on the display device 130 and makes a pathology diagnosis.

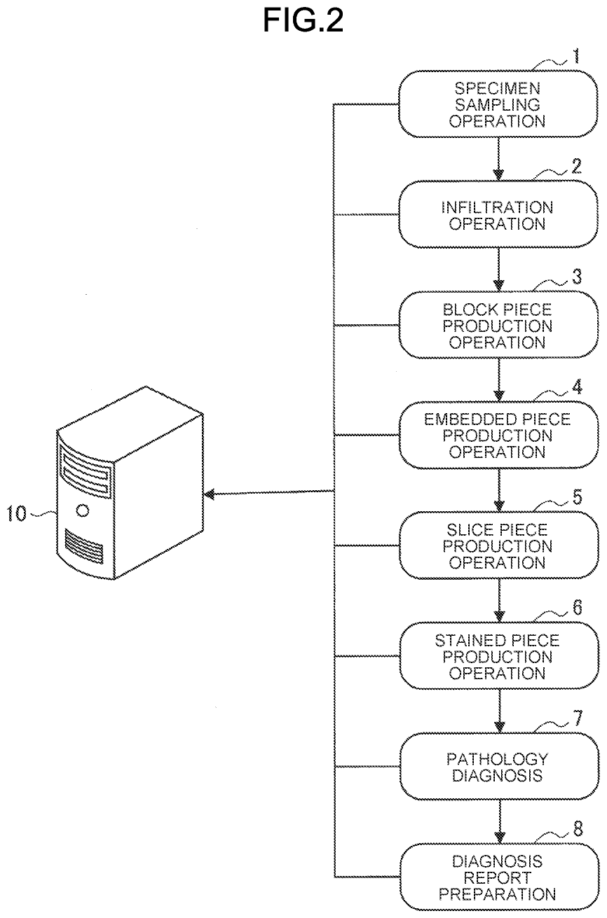

With reference to FIG. 2, the effect that the operation work of the operator is reduced by the medical support system 1000 will be described specifically. FIG. 2 is a diagram illustrating a process of pathology diagnosis in the case where the technique of the disclosure is not used.

As illustrated in FIG. 2, in each of the operations 1 to 6, as in FIG. 1, an operator checks the orientation of a specimen with respect to a pathology slide, specimen information on the specimen that is processed, or the like, and inputs it to the hospital information system 10, or the like. After the stained piece production operation 6 ends, a pathologist performs pathology diagnosis 7 on the stained piece with a medical microscope. When the pathology diagnosis 7 ends, the pathologist performs a diagnosis report preparation operation 8 of inputting diagnosis results together to the hospital information system 10, or the like.

In the medical microscope in the pathology diagnosis 7, a pathology slide containing a stained piece is placed in a certain position appropriate to perform pathology diagnosis. As for a stained piece that is placed in a position inappropriate to perform pathology diagnosis, re-processing from the specimen may be performed.

The medical support system 1000 is capable of acquiring a digitized image of a stained piece by capturing an image of the stained piece and thus the pathologist is able to freely rotate the image of the stained piece and make a pathology diagnosis. Thus, an operator is able to appropriately pass a specimen to a pathologist appropriately without performing the operation of checking the orientation of the specimen on a pathology slide.

The medical support system 1000 is able to automatically associate specimens in the respective operations, which makes it possible to promptly check whether specimens are not mixed up. Accordingly, the operator is able to reduce the time taken by a checking operation.

### 2. Embodiment

2.1. Configuration

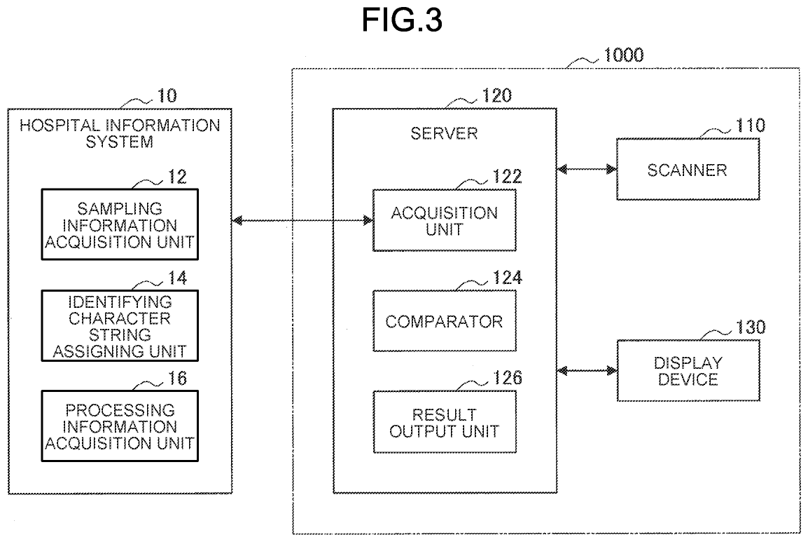

With reference to FIGS. 3 to 9, functions and a configuration of the medical support system 1000 will be described. FIG. 3 is a block diagram to describe configurations of the medical support system 1000 and the hospital information system 10 that outputs and inputs various types of information to and from the medical support system 1000. Before describing the medical support system 1000, the hospital information system 10 will be described. The configuration of the medical support system 1000 is not limited to the example and each function and the configuration may be present as being aggregated in a single device or may be distributed to multiple devices.

The hospital information system 10 refers to a consolidating that tracks and manages a specimen in each of the operations 1 to 6 like those illustrated in FIG. 2. The hospital information system 10 includes a sampling information acquisition unit 12, an identifying character string assigning unit 14, and a processing information acquisition unit 16.

The sampling information acquisition unit 12 has a function of acquiring sampling information on a specimen that is sampled from a living body. Specifically, the sampling information acquisition unit 12 acquires information that is input when a specimen is accepted. For example, the sampling information acquisition unit 12 acquires the content of an examination request on the specimen. The content of the examination request includes information on a patient from which the specimen is sampled, the name of organ of the specimen, and the number of specimens to be sampled from the patient. The sampling information acquisition unit 12 also acquires information that is acquired by such an operation of infiltrating a specimen with an organic solvent to prevent tissue or cells of the specimen from denaturing or decaying as that illustrated in the operation 2. The sampling information includes information, such as a physician who provides treatment, another physician who is involved in the specimen, the condition of an inpatient or an outpatient, insurance information, a requested examination, the examination condition, and the examination result.

The identifying character string assigning unit 14 has a function of assigning an identifying character string to a specimen and specimen information when the specimen information on the specimen is input to the sampling information acquisition unit 12. The identifying character string is a character string that associates the specimen and the specimen information with each other and the same identifying character string is assigned to the specimen and the specimen information on the specimen. The identifying character string may be formed of characters or numbers or a character string of a mixture of letters and numbers. In the embodiment, the example in which the identifying character string assigning unit 14 assigns letters or numbers is taken as an example; however, the identifying character string assigning unit 14 may assign an identifier other than letters and numbers, such as a barcode or a two-dimensional code.

The processing information acquisition unit 16 has a function of acquiring information on processing of the specimen that is obtained in each of the steps 3 to 6 like those illustrated in FIG. 1. Specifically, the processing information acquisition unit 16 acquires information on at least any one of a block piece obtained by cutting a block out of the specimen, an embedded piece in which the block piece is embedded, a slice piece obtained by slicing the embedded piece, and a stained piece obtained by staining the slice piece.

The operation of producing a block piece includes an operation of diagnosing mainly the site, size, properties, breadth, etc., of a lesion of the specimen and then producing a part necessary for diagnosis as a sample. The processing information acquisition unit 16 may acquire information that is acquired in such an operation of producing a block piece.

The embedded piece production operation includes an operation of embedding tissue in paraffin (wax) to produce an embedded paraffin block. The processing operation for an embedded piece includes an operation of replacing water with paraffin wax, that is, an operation of turning soft and wet tissue into a solid paraffin block, then embedding the tissue in another cassette containing much more soluble wax, and cooling and fixing the tissue. The processing information acquisition unit 16 may acquire information that is acquired in such an operation of producing an embedded piece.

The slice piece production operation includes an operation of slicing the paraffin block into a 3 to 4 mm slice with, for example, a machine referred to as microtome, attaching the slice onto a slide glass and drying the slice. The processing information acquisition unit 16 may acquire information that is acquired by such an operation of producing a slice piece.

The stained piece production operation includes an operation of staining the specimen such that the specimen is observable. The stained piece processing operation performs staining by a known pigment and staining selectively shows the tissue structure clearly and clarify the form of the specimen. The processing information acquisition unit 16 may acquire information that is acquired by such an operation of producing a stained piece.

The processing information that is acquired in each operation contains a captured image obtained by capturing an image of the specimen in each operation. The processing information further contains information that is input to the hospital information system 10 in each operation, or information on determination on whether a further operation is required in the operation process. The hospital information system 10 consolidates such specimen information on the specimen as that described above.

The functions and configuration of the hospital information system 10 have been described. The medical support system 1000 will be described next.

The medical support system 1000 includes the scanner 110, the server 120, and the display device 130. The medical support system 1000 compares evaluation piece information that is acquired by the scanner 110 and such specimen information acquired from the hospital information system 10 as that described above and presents a comparison result to a user, using the display device 130.

First of all, the scanner 110 will be described. The scanner 110 has a function of scanning an evaluation piece into which a specimen is processed and acquiring an evaluation piece image. The evaluation piece refers to a specimen that is produced in the stained piece production operation 6 and on which the scanning operation is performed by the scanner 110. The evaluation piece image is an image that is obtained by capturing an image of the evaluation piece and that contains an evaluation piece identifying character string.

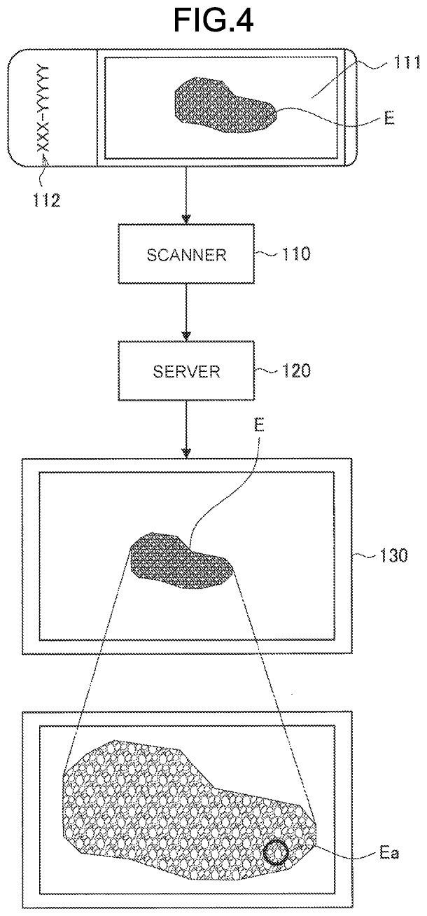

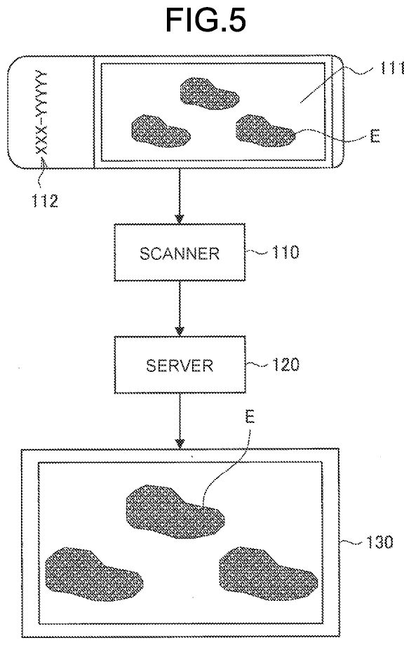

With reference to FIGS. 4 and 5, how the scanner 110 scans an evaluation piece will be described. FIGS. 4 and 5 are diagrams each illustrating an example in which an image of an evaluation piece is captured by the scanner and the image is displayed on the display device.

As illustrated in FIG. 4, an image of a pathology slide 111 containing an evaluation piece E and an evaluation piece identifying character string 112 that is assigned to the pathology slide 111 is captured by the scanner 110. Accordingly, the medical support system 1000 acquires an evaluation piece image. The acquired evaluation piece image is processed by the server 120 to be described below and is then presented by the display device 130 to the user.

The scanner 110 is capable of acquiring information on a cell array Ea in the evaluation piece E from the evaluation piece image. In other words, the scanner 110 is capable of acquiring information on the cell array Ea by acquiring the evaluation piece in a high resolution. The information on the cell array Ea is, for example, a characteristic cell array that differs according to each organ. As in the case of the method of acquiring a captured image that is illustrated in FIG. 4, the scanner 110 is capable of acquiring information on the number of evaluation pieces E that are arranged on the pathology slide 111 by capturing an image of the pathology slide 111 containing the evaluation pieces and an evaluation piece identifying character string 112 that is assigned to the pathology slide 111 as illustrated in FIG. 5. The scanner 110 is able to acquire not only the evaluation piece image but also evaluation piece information containing evaluation piece information containing the cell array Ea or the number of evaluation pieces E that is acquired from the evaluation piece image.

The server 120 includes an acquisition unit 122, a comparator 124, and a result output unit 126. The server 120 has a function of comparing the specimen information on the specimen that is acquired by the acquisition unit 122 and the evaluation piece information that is acquired from the evaluation piece image and outputting the evaluation result.

The acquisition unit 122 has a function of acquiring evaluation piece information containing an evaluation piece image that is acquired by the scanner 110. The acquisition unit 122 further has a function of acquiring specimen information necessary for a process in the comparator 124 from the hospital information system 10. The acquisition unit 122 may keep being connected or be connected as required to the scanner 110 to input and output evaluation piece information. The acquisition unit 122 may keep being connected or be connected as required to the hospital information system 10 to input and output specimen information or evaluation piece information.

The comparator 124 has a function of comparing the specimen information and the evaluation piece information that are acquired by the acquisition unit 122. Specifically, the comparator 124 compares specimen information and evaluation piece information whose specimen identifying character string and evaluation piece identifying character string represent the same character string. In other words, the comparator 124 compares specimen information on a specimen at each operation stage in the hospital information system, which is specimen information having the same character string as that of an evaluation piece identifying character string acquired from an evaluation piece image, and evaluation piece information with each other.

The comparator 124 may calculate a degree of similarity between a specimen and an evaluation piece as the aforementioned comparison result. A degree of similarity may be an index representing a degree representing how much the specimen information and the evaluation piece to which the same identifying character string is assigned are similar to each other.

For example, the comparator 124 may calculate a degree of similarity by comparing the shape of the evaluation piece and the shape of the specimen. The shapes may be the shapes of the surfaces of the evaluation piece and the specimen or may be the shapes further having depths with respect to the surfaces. A known shape comparison unit can be used as a unit that compares the shapes.

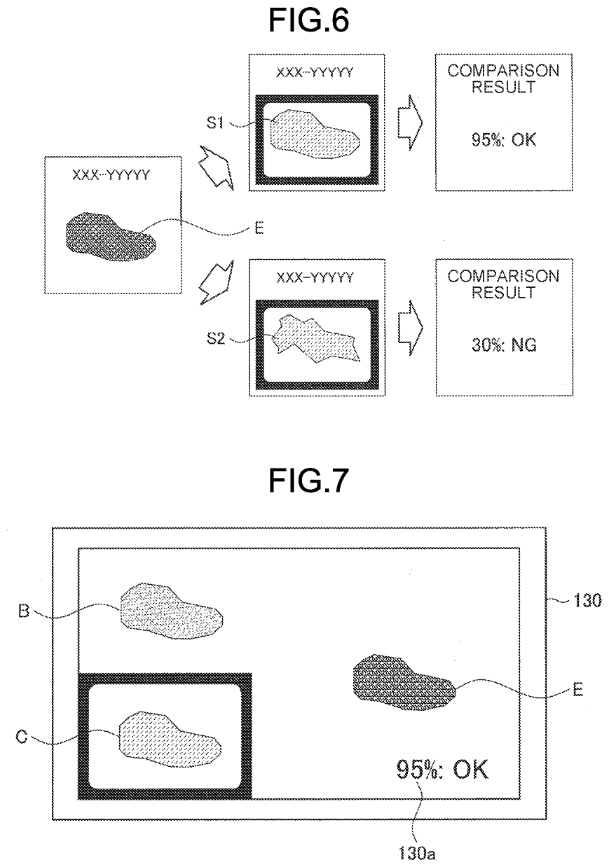

With reference to FIG. 6, comparison by the comparator 124 between the shape of an evaluation piece and the shape of a specimen will be described. The comparator 124 selects an evaluation piece E that is specified by a user, or the like, and that is used for pathology diagnosis. The comparator 124 acquires specimen information that is associated with a specimen identifying character string (XXX-YYYYY) having the same character string as that of an evaluation piece identifying character string (XXX-YYYYY) of the selected evaluation piece E and compares the shape of the specimen S1 contained in the specimen information and the shape of the evaluation piece E.

The comparator 124 calculates an agreement rate between the shape of the specimen S1 and the shape of the evaluation piece E as the aforementioned comparison result. The agreement rate is an index representing a degree at which the shape of the specimen and the shape of the evaluation piece agree with each other and, for example, the agreement rate may be a numerical value obtained by calculating, when the shape of the specimen and the shape of the evaluation piece are superimposed, how much they overlap. For example, FIG. 6 illustrates that the agreement rate between the specimen S1 and the evaluation piece E is 95%.

When the calculated agreement rate is at or above a threshold, the comparator 124 may certify that the evaluation piece is an evaluation piece into which the specimen is processed. For example, when the threshold is 80% in FIG. 6, because the agreement rate between the specimen S1 and the evaluation piece E is 95% and is at or above the threshold, the comparator 124 certifies that the evaluation value E is an evaluation piece into which the specimen S1 is processed and outputs “OK”. The comparator 124 accordingly outputs a result of certifying the evaluation piece, using the agreement rate, and thus the operator need not check by sight the agreement rate between the specimen S1 and the evaluation piece E and the operation work reduces. Note that the threshold is preferably a threshold that makes it possible to determine whether they are the evaluation piece and the specimen that is identical with the evaluation piece and certify that they are the evaluation piece and the specimen that is identical with the evaluation piece and the threshold may be determined as appropriate.

On the other hand, when a processing error or mixing up of specimens occurs at the stage of processing the specimen into an evaluation piece, there is a possibility that the specimen information having the same character string as the evaluation piece identifying character string may contain information on a specimen different from that of the evaluation piece. In this case, the comparator 124 compares the shape of the evaluation piece E and the shape of another specimen S2 not corresponding to the evaluation piece E. In this case, for example, the comparator 124 calculates an agreement rate lower than that of the specimen S1. When the threshold is set at 80% as described above, because the agreement rate between the specimen S2 and the evaluation piece E is 30% and is under the threshold, the comparator 124 certifies that the evaluation piece E is not an evaluation specimen into which the specimen S2 is processed and outputs “NG”.

The comparator 124 may calculate an agreement rate based on not only the above-described comparison in shape but also the specimen information and the evaluation piece information other than the shapes. Specifically, an agreement rate may be calculated by comparing information of the number of specimens that are processed into evaluation pieces and information of the number of evaluation pieces to each other. When the number of evaluation pieces into which the specimen is processed and the number of evaluation pieces acquired from the evaluation piece image are compared to each other and the numbers differ from each other, the specimen information and the evaluation piece information highly likely do not agree with each other. Thus, the comparator 124 may calculate an agreement rate by not only making comparison in shape but also making comparison in information, such as the number of evaluation pieces, etc. In this case, the display device 130 may improve identifiability of the number of evaluation pieces by assigning numbers to the respective evaluation pieces displayed and presenting the numbers to the user.

An agreement rate may be calculated by comparing information of the name of the organ of the specimen and the evaluation piece information. The tissue array of the evaluation piece that can be known from the evaluation piece image may differ depending on the organ. Thus, by comparing the information of the name of the organ of the specimen and the evaluation piece information and thus comparing the name of the organ of the specimen and the evaluation piece information, an agreement rate may be calculated. The comparator 124 outputs such a comparison result as the above-described one to the result output unit 126. The example in which an agreement rate is calculated by the medical support system 1000 is described above. The embodiment is not limited to the above-described example and a doctor may compare the evaluation piece image and any one of sets of specimen information, such as the number of specimens, the name of the organ, the tissue array, thereby determining whether the evaluation piece and the specimen agree with each other. Note that comparison between information of the name of the organ of the specimen and the evaluation piece information may be made by the medical support system 1000 or the operator.

The result output unit 126 outputs the comparison result that is obtained by the comparator 124 to the display device 130. The comparison result specifically represents the agreement rate, the certifying result, etc. The result output unit 126 has a function of outputting the result in a changed display mode, using the display device 130.

The display device 130 has a function of displaying the result that is output from the result output unit 126. For example, the display device 130 displays the image of the processed specimen, the evaluation piece image, the calculated agreement rate, and the certifying result.

The display device 130 is formed of a device capable of auditorily notifying the user of the acquired information. There is, as such a device, a display device, such as a CRT display device, a liquid crystal display device, a plasma display device, an EL display device, a laser projector, or a LED projector and a lamp. Specifically, the display device 130 displays the specimen image and the evaluation piece image. The display device 130 further visually displays the comparison result obtained by various types of processing performed by the server 120 in various forms, such as a text, an image, a table, and a graph.

With reference to FIGS. 7 to 9, the example in which the result that is output by the result output unit 126 is presented to the user will be described specifically. FIGS. 7 to 9 are diagrams illustrating examples of presentation of the comparison result by the display device 130. As illustrated in FIG. 7, on the display device 130, the evaluation piece image of the evaluation piece E is displayed on one side of a display area and a specimen B and a specimen C serving as subjects to be compared to the evaluation piece E are arrayed vertically and displayed on the other side of the display area. For example, the specimen B may be a slice piece and the specimen C may be an embedded piece. FIG. 7 displays the two specimens as subjects of comparison but the number of specimens serving as subjects of comparison is not limited to this.

In FIG. 7, a display 130a representing the agreement rate and the certifying result is represented under the image of the evaluation piece D. The agreement rate and the certifying result that are represented on the display 130a may be an agreement rate and a certifying result of a single specimen among the specimens that are displayed on the other side of the display area. When multiple specimens are displayed, agreement rates and certifying results of the respective specimens may be arrayed on the display 130a. An integrated agreement rate obtained by integrally calculating an agreement rate with each specimen may be displayed on the display 130a.

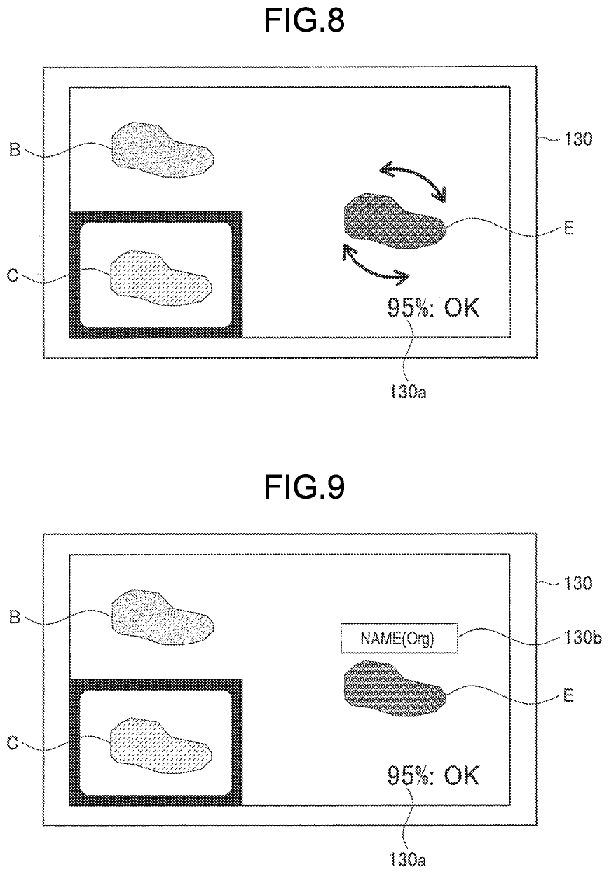

In FIG. 8, as in FIG. 7, the specimen B, the specimen C, the evaluation piece E and the display 130a are displayed. On the display device 130, the evaluation piece E may be displayed rotatably. Based on the orientation of the specimen B or the specimen C, the evaluation piece E may be rotated automatically and presented to the user. This makes it is possible to make a pathology diagnosis with the orientation being aligned between the evaluation piece E and the specimen B or the specimen C. In the process of processing the specimen, a practitioner performs the operation of processing the specimen in consideration of the three-dimensional shape or orientation and, also in pathology diagnosis, the pathologist performs pathology diagnosis in consideration of the three-dimensional shape or the orientation. For example, the practitioner or the pathologist performs operations and diagnosis in consideration of information on in which direction a cancer is invading or in which direction a biopsy needle is operated. The operator is thus operating in consideration of the orientation when producing an embedded piece in each operation process and accordingly much time is required for the operation. As illustrated in FIG. 8, for example, making the evaluation piece E rotatable makes it possible to rotate the evaluation piece E according to the orientation of the specimen B or the specimen C and make a pathology diagnosis, which makes it possible to reduce the time to take the orientation into consideration in the process of processing the specimen.

In FIG. 9, as in FIG. 7, the specimen B, the specimen C, the evaluation piece E, and the display 130a are displayed and a display 130b representing the details of the specimen information is displayed above the evaluation piece E. The details of the specimen information may be, for example, details of the specimen information having the same character string as the evaluation piece identifying character string of the evaluation piece E. Specifically, the name of the organ of the specimen may be displayed. Displaying the name of the organ of the specimen and the evaluation piece image enables the pathologist to check the evaluation piece information, such as the tissue array of the evaluation piece that can be known from the evaluation piece image, and the name of the organ. Accordingly, the pathologist is able to make a pathology diagnosis with more certainty.

The configuration and functions of the medical support system 1000 have been described.

2.2. Operation Flow

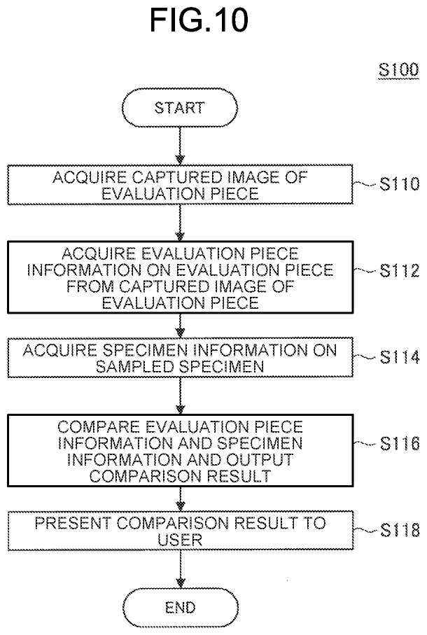

In this term, an operation flow (S100) of the medical support system 1000 will be described. FIG. 10 is a chart illustrating an example of a flow of operations of the medical support system 1000.

First of all, the scanner 110 captures an image of an evaluation piece and acquires the captured image of the evaluation piece (S110).

The acquisition unit 122 acquires evaluation piece information on the evaluation piece from the captured image of the evaluation piece (S112).

The acquisition unit 122 acquires specimen information on a specimen that is sampled (S114).

The comparator 124 compares the evaluation piece information and the specimen information and outputs a comparison result (S116).

The result output unit 126 presents the comparison result to the user via the display device 130 (S118).

The medical support system 1000 is able to acquire a digitized evaluation piece image because each component performs the above-described operation flow. This makes the evaluation piece image rotatable and makes it possible reduce the operation work of the operator in the specimen processing operation, such as performing operations in consideration of the orientation of the specimen.

### 3. Modification

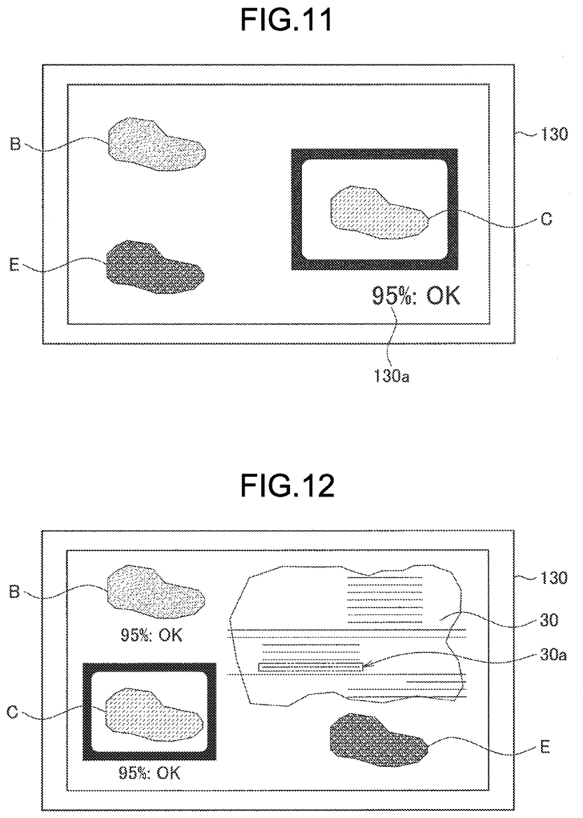

The medical support system 1000 according to the embodiment has been described. In this term, with reference to FIGS. 11 to 14, an example of modification of the medical support system 1000 according to the embodiment will be described. FIGS. 11 to 14 are diagrams to describe examples of modification of the medical support system 1000 according to the embodiment.

Modification 1

In the above-described embodiment, the example in which the evaluation piece E, the agreement rate, and the display 130a of the certifying result are displayed on one side of the display area is escribed. As a modification of the above-described embodiment, as illustrated in FIG. 11, the specimen B and the evaluation piece E may be displayed vertically on one side of the display area (on the left side in FIG. 11) and the specimen C, and the display 130a of the agreement rate and the certifying result may be displayed on the other side (on the right side in FIG. 11).

Modification 2

In the above-described embodiment, the example in which the specimen B, the specimen C, the evaluation piece E, and the display 130a of the agreement rate and the certifying result are displayed on the display area is described. As a modification of the embodiment, as illustrated in FIG. 12, a captured image of a block piece 30 that is cut out of a specimen that is sampled from a living body is displayed and cut-out lines along which multiple evaluation pieces are cut out are illustrated in the block piece 30 are illustrated. In the modification, a cut-out line 30a may be displayed as a position corresponding to the evaluation piece E that is displayed on the display area. Displaying the cut-out line 30a as described above enables a user to instantly determine where the evaluation piece E is positioned in the block piece 30, which makes it possible to increase the amount of information to perform pathological diagnosis.

Modification 3

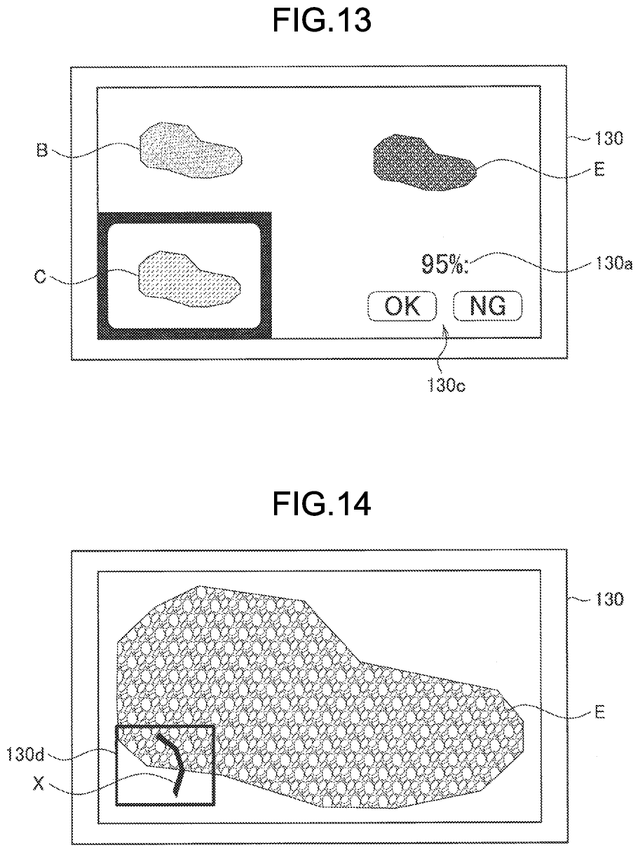

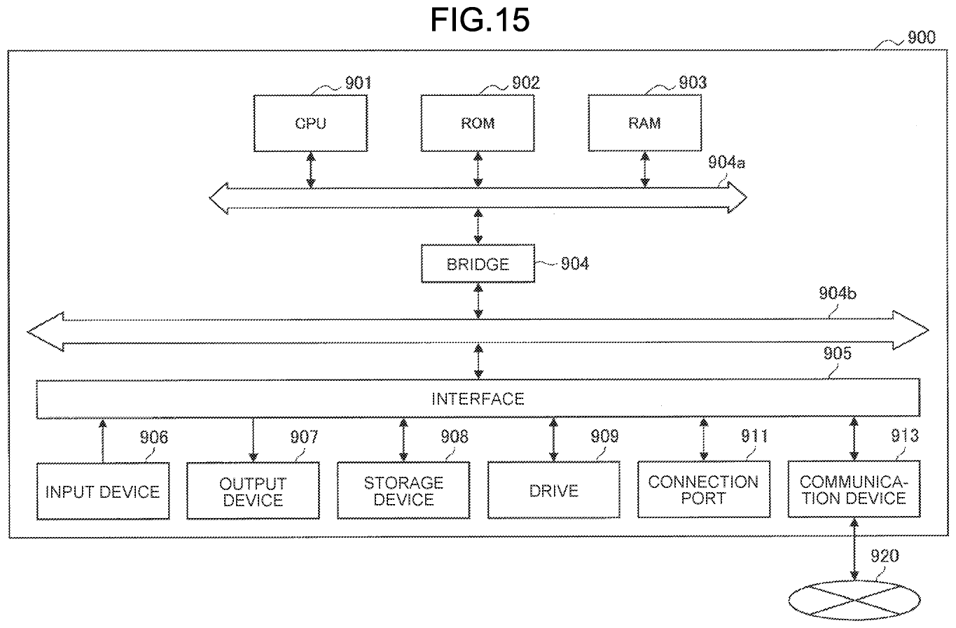

In the above-described embodiment, the example in which the agreement rate and the certifying result based on the agreement rate among the comparison result that is output from the server 120 is displayed by the display device 130 has been described. As a modification of the above-described embodiment, as illustrated in FIG. 13, the agreement rate that is calculated by the comparator 124 and an input button 130c for the user to input the certifying result with reference to the agreement rate may be displayed. Accordingly, the practitioner determines the certifying result, which enables flexible comparison.

When the practitioner inputs the certifying result as “OK”, the medical support system 1000 regards that preparation for pathology diagnosis has completed and notify the pathologist of completion of preparation for pathology diagnosis. When the practitioner inputs the certifying result as “NG”, the operator who processes specimens may be notified that mixing up has occurred in processing specimens. Such notification enables a pathologist or a practitioner to engage in diagnosis or operations without wasting time. As for such notification, the medical support system 1000 may select a preferable pathologist from multiple pathologists in consideration of difficulty in evaluating a specimen or an evaluation piece, specialty of the pathologist, crowdedness of tasks, etc., and transmits a notification to the pathologist. When multiple pathologists make diagnoses, the medical support system 1000 may transmit notifications in consideration of a circulation route of the pathologists. Difficulty in evaluating a specimen or an evaluation piece may be determined by AI (Artificial Intelligence). Difficulty in evaluating a specimen or an evaluation piece includes difficulty in making a pathology diagnosis.

Modification 4

In the above-described embodiment, the example in which the comparison result obtained by comparing the evaluation piece information obtained from the evaluation piece E and the specimen information with each other is output is described. As a modification of the embodiment, as illustrated in FIG. 14, the medical support system 1000 may detect a foreign matter X that is contained in the evaluation piece image and present the foreign matter X to the user. For example, as a display method, a frame display 130d surrounding the foreign matter X may be displayed. The foreign matter X may be an object, such as a dust or a bubble, unnecessary in making a pathology diagnosis.

In another example, in addition to detection of a foreign matter, the medical support system 1000 may detect a defect of the evaluation piece in shape including folding of the evaluation piece E and present the defect to the user. The folding of the evaluation piece E is detectable in consideration of linearity of the evaluation piece E, etc.

In the above-described embodiment, the example in which the medical support system 1000 includes the scanner 110, the server 120 and the display device 130 is described;

however, embodiments are not limited thereto. Each configuration and function of the medical support system 1000 described in the embodiment described above, for example, may be consolidated and installed in the scanner 110.

In the above-described embodiment and modifications, the medical support system 1000 including the scanner 110, the server 120 and the display device 130 is described; however, embodiments are not limited thereto. For example, the functions of the configurations of the medical support system 1000 may be consolidated and installed in the scanner 110.

### 4. Hardware Configuration

With reference to FIG. 18, an example of a hardware configuration of a server included in the medical support system according to the above-described embodiment, or the like, will be described. FIG. 18 is a block diagram illustrating the example of the hardware configuration of the server according to the above-described embodiment, or the like.

As illustrated in FIG. 18, an information processing device 900, such as the server included in the medical support system, includes a CPU (Central Processing Unit) 901, a ROM (Read Only Memory) 902, a RAM (Random Access Memory) 903, and a host bus 904a. The information processing device 900 includes a bridge 904, an external bus 904b, an interface 905, an input device 906, a display device 907, a storage device 908, a drive 909, a connection port 911, and a communication device 913. The information processing device 900 may include, instead of or in addition to the CPU 901, a processing circuit, such as an electric circuit, DSP or an ASIC.

The CPU 901 functions as a processing unit and a control device and controls general operations in the information processing device 900 according to various programs. The CPU 901 may be a microprocessor. The ROM 902 stores programs and operational parameters that the CPU 901 uses. The RAM 903 temporarily stores programs that are used in execution by the CPU 901 and parameters that appropriately change in the execution. The CPU 901 may, for example, form the comparator 124 of the server 120 that is illustrated in FIG. 3.

The CPU 901, the ROM 902 and the RAM 903 are connected with one another via the host bus 904a including a CPU bus, or the like. The host bus 904a is connected to the external bus 904b, such as a PCI (Peripheral Component Interconnect/Interface) bus via the bridge 904. Note that the host bus 904a, the bridge 904 and the external bus 904b need not necessarily be configured separately and a single bus may implement these functions.

The input device 906 is implemented by a device, such as a mouse, a keyboard, a touch panel, a button, a microphone, a switch, or a lever, with which a user inputs information. The input device 906 may be, for example, a remote control device that uses infrared rays or other radio waves or may be an external connection device, such as a mobile phone or a PDA corresponding to operations on the information processing device 900. The input device 906 may, for example, include an input control circuit that generates an input signal based on information that is input by the user with the aforementioned input unit and outputs the input signal to the CPU 901. The user of the information processing device 900 is able to input various types of data and issues an instruction of a processing operation to the information processing device 900 by operating the input device 906.

The display device 907 is formed of a device capable of visually or auditorily notifying the user of acquired information. There are, as such a device, a display device, such as a CRT display device, a liquid crystal display device, a plasma display device, an EL display device, a laser projector, or an LED projector and a lamp, or an audio output device, such as a speaker or headphones. The display device 907, for example, outputs a result that is obtained by various types of processing performed by the information processing device 900. Specifically, the display device 907 visually displays the result obtained by the various types of processing performed by the information processing device 900 in various forms, such as a text, an image, a table and a graph. On the other hand, when the audio output device is used, an audio signal formed of reproduced audio data or acoustic data is converted into an analog signal and the analog signal is output auditorily. The display device 907 is, for example, the display device 130 that is illustrated in FIG. 3.

The storage device 908 is a device for storing data that is formed as an example of the storage of the information processing device 900. The storage device 908 is, for example, implemented by a magnetic storage device, such as an HDD, a semiconductor storage device, an optical storage device, or a magneto-optical storage. The storage device 908 may include a storage medium, a recording device that records data in the recording medium, a read device that reads data from the storage medium, and a deletion device that deletes data that is recorded in the recording medium. The storage device 908 stores programs that are executed by the CPU 901, various types of data, and various types of data acquired from outside. The storage device 908, for example, stores captured images that are used by the server 120 illustrated in FIG. 3 to control the display device 130 or various parameters that are used to generate a comparison result of information that is acquired from the captured images.

The drive 909 is a storage media reader/writer and is incorporated in or externally attached to the information processing device 900. The drive 909 reads information that is stored in a removable storage medium, such as a magnetic disk, an optical disk, an electro-magnetic disk or a semiconductor memory, that is attached to the drive 909 and outputs the information to the RAM 903. The drive 909 is able to write information in the removable storage medium.

The connection port 911 is an interface that is connected to an external device and is, for example, a connection port to an external device to which data can be transmitted via, for example, an USB (Universal Serial Bus).

The communication device 913 is a communication interface formed of a communication device, or the like, for connecting to a network 920. The communication device 913 is, for example, a communication card for a wired or wireless LAN (Local Area Network), LTE (Long Term Evolution), Bluetooth (trademark) or WUSB (Wireless USB). The communication device 913 may be a router for optical communication, a router for ADSL (Asymmetric Digital Subscriber Line), a modem for various types of communication, or the like. The communication device 913, for example, is able to transmit and receive signals on the Internet or with another communication device according to a given protocol, such as TCP/IP. For example, the server 120 and the scanner 110 illustrated in FIG. 3 may input and output various types of information via the communication device 913.

The network 920 is a wired or wireless transmission path for information that is transmitted from a device that is connected to the network 920. For example, the network 920 may include a public network, such as the Internet, a telephone network or a satellite network, and various types of LAN (Local Area Network) including Ethernet (trademark), and a WAN (Wide Area Network). The network 920 may include a dedicated network, such as an IP-VPN (Internet Protocol-Virtual Private Network).

The information processing device 900 is able to produce a computer program for enabling hardware, such as the CPU, the ROM and the RAM, incorporated in the medical support system 1000 to implement the same functions as those of each configuration of the medical support system 1000 according to the above-described embodiment. A recording medium in which the computer program is stored is within the scope of the technique according to the disclosure.

The preferable embodiment of the disclosure has been described in detail with reference to the accompanying drawings; however, the technical scope of the disclosure is not limited to the example. It is obvious that those with general knowledge in the technical field of the disclosure can reach various modification examples or correction examples within the scope of the technical idea described in the claims and it is understood that they naturally belong to the technical scope of the disclosure.

The effects disclosed herein are explanatory and exemplary only and thus are not definitive. In other words, the technique according to the disclosure can achieve, in addition to the above-described effects or instead of the above-described effects, other effects obvious to those skilled in the art from the description herein.

The following configuration also belongs to the technical scope of the disclosure.

### Claims

(1) A medical support system comprising:

a scanner configured to scan an evaluation piece into which a specimen is processed and acquire an evaluation piece image;

a server configured to compare specimen information on the specimen and evaluation piece information that is obtained from the evaluation piece image and output a comparison result; and

a display device configured to present the comparison result to a user.

(2) The medical support system according to the above (1), wherein the specimen information contains information on processing of the specimen into the evaluation piece.
(3) The medical support system according to the above (2), wherein the information on the processing contains information on at least any one of a block piece obtained by cutting a block out of the specimen, an embedded piece in which the block piece is embedded, a slice piece obtained by slicing the embedded piece, and a stained piece obtained by staining the slice.
(4) The medical support system according to any one of the above (1) to (3), wherein

the specimen information contains a specimen identifying character string that identifies the specimen,

the scanner is configured to capture an image of an evaluation piece identifying character string that is assigned to the evaluation piece and the whole evaluation piece, and

the server is configured to compare the specimen and the evaluation piece with the specimen identifying character string and the evaluation piece identifying character string representing the same character string and output the comparison result.

(5) The medical support system according to any one of the above (1) to (3), wherein the server is configured to compare the specimen information and the evaluation piece information and output, as the comparison result, a degree of similarity between the specimen and the evaluation piece.
(6) The medical support system according to any one of the above (1) to (5), wherein the server is configured to compare a shape of the evaluation piece into which the specimen is processed and a shape of the processed specimen and output the comparison result.
(7) The medical support system according to the above (3), wherein the server is configured to calculate an agreement rate between the shape of the specimen and the shape of the evaluation piece as the comparison result and, when the agreement rate is at or above a threshold, certify that the evaluation piece is an evaluation piece into which the specimen is processed.
(8) The medical support system according to the above (7), wherein the server is configured to further calculate the agreement rate based on the specimen information and the evaluation piece information.
(9) The medical support system according to the above (8), wherein the specimen information contains information of the number of the evaluation pieces into which the specimen is processed and the evaluation piece information contains information of the number of the evaluation pieces.
(10) The medical support system according to the above (8), wherein the specimen information contains information on a name of an organ of the specimen.
(11) The medical support system according to the above (7), wherein the display device is configured to display an image of the processed specimen, the evaluation piece image, the calculated agreement rate, and a result of the identifying.
(12) The medical support system according to the above (11), wherein the evaluation image is displayed rotatably.
(13) The medical support system according to the above (12), wherein an orientation in which the evaluation piece image is displayed is determined based on the image of the processed specimen.
(14) The medical support system according to the above (11) or (12), wherein the display device is configured to further display a position of the evaluation piece in the block piece.
(15) The medical support system according to the above (11), wherein, based on the result of the identifying, completion of preparation of the evaluation piece is presented to a pathologist that is selected from multiple pathologists based on at least one of difficulty in evaluating the evaluation piece, specialty of the pathologist, and crowdedness of tasks of the pathologist.
(16) The medical support system according to the above (15), wherein, the difficulty in evaluating the evaluation piece is determined by artificial intelligence.
(17) The medical support system according to any one of the above (11) to (14), wherein, the display device is configured to detect a foreign matter that is contained in the evaluation piece image and present a result of detecting the foreign matter to a user.
(18) The medical support system according to any one of the above (1) to (17), wherein the evaluation piece is used for pathology diagnosis.
(19) A medical support device comprising:

an acquisition unit configured to scan an evaluation piece into which a specimen is processed and acquire an evaluation piece image;

a comparison result output unit configured to compare specimen information on the specimen and evaluation piece information that is obtained from the evaluation piece image and output a comparison result; and

a display unit configured to present the comparison result to a user.

(20) A medical support method comprising:

by a scanner, scanning an evaluation piece into which a specimen is processed and acquiring an evaluation piece image;

by a calculation device, comparing specimen information on the specimen and evaluation piece information that is obtained from the evaluation piece image and outputting a comparison result; and

by a display device, presenting the comparison result to a user.

### REFERENCE SIGNS LIST

1 SPECIMEN SAMPLING OPERATION
2 INFILTRATION OPERATION
3 BLOCK PIECE PRODUCTION OPERATION
4 EMBEDDED PIECE PRODUCTION OPERATION
5 SLICE PIECE PRODUCTION OPERATION
6 STAINED PIECE PRODUCTION OPERATION
10 HOSPITAL INFORMATION SYSTEM
12 SAMPLING INFORMATION ACQUISITION UNIT
14 IDENTIFYING CHARACTER STRING ASSIGNING UNIT
16 PROCESSING INFORMATION ACQUISITION UNIT
110 SCANNER
120 SERVER
122 ACQUISITION UNIT
124 COMPARATOR
126 RESULT OUTPUT UNIT
130 DISPLAY DEVICE

1. A medical support system comprising:
a scanner configured to scan an evaluation piece into which a specimen is processed and acquire an evaluation piece image;
a server configured to compare specimen information on the specimen and evaluation piece information that is obtained from the evaluation piece image and output a comparison result; and
a display device configured to present the comparison result to a user.

2. The medical support system according to claim 1, wherein the specimen information contains information on processing of the specimen into the evaluation piece.

3. The medical support system according to claim 2, wherein the information on the processing contains information on at least any one of a block piece obtained by cutting a block out of the specimen, an embedded piece in which the block piece is embedded, a slice piece obtained by slicing the embedded piece, and a stained piece obtained by staining the slice.

4. The medical support system according to claim 3, wherein
the specimen information contains a specimen identifying character string that identifies the specimen,
the scanner is configured to capture an image of an evaluation piece identifying character string that is assigned to the evaluation piece and the whole evaluation piece, and
the server is configured to compare the specimen and the evaluation piece with the specimen identifying character string and the evaluation piece identifying character string representing the same character string and output the comparison result.

5. The medical support system according to claim 3, wherein the server is configured to compare the specimen information and the evaluation piece information and output, as the comparison result, a degree of similarity between the specimen and the evaluation piece.

6. The medical support system according to claim 4, wherein the server is configured to compare a shape of the evaluation piece into which the specimen is processed and a shape of the processed specimen and output the comparison result.

7. The medical support system according to claim 6, wherein the server is configured to calculate an agreement rate between the shape of the specimen and the shape of the evaluation piece as the comparison result and, when the agreement rate is at or above a threshold, certify that the evaluation piece is an evaluation piece into which the specimen is processed.

8. The medical support system according to claim 7, wherein the server is configured to further calculate the agreement rate based on the specimen information and the evaluation piece information.

9. The medical support system according to claim 8, wherein the specimen information contains information of the number of the evaluation pieces into which the specimen is processed and the evaluation piece information contains information of the number of the evaluation pieces.

10. The medical support system according to claim 8, wherein the specimen information contains information on a name of an organ of the specimen.

11. The medical support system according to claim 7, wherein the display device is configured to display an image of the processed specimen, the evaluation piece image, the calculated agreement rate, and a result of the identifying.

12. The medical support system according to claim 11, wherein the evaluation image is displayed rotatably.

13. The medical support system according to claim 12, wherein an orientation in which the evaluation piece image is displayed is determined based on the image of the processed specimen.

14. The medical support system according to claim 12, wherein the display device is configured to further display a position of the evaluation piece in the block piece.

15. The medical support system according to claim 11, wherein, based on the result of the identifying, completion of preparation of the evaluation piece is presented to a pathologist that is selected from multiple pathologists based on at least one of difficulty in evaluating the evaluation piece, specialty of the pathologist, and crowdedness of tasks of the pathologist.

16. The medical support system according to claim 15, wherein, the difficulty in evaluating the evaluation piece is determined by artificial intelligence.

17. The medical support system according to claim 11, wherein, the display device is configured to detect a foreign matter that is contained in the evaluation piece image and present a result of detecting the foreign matter to a user.

18. The medical support system according to claim 1, wherein the evaluation piece is used for pathology diagnosis.

19. A medical support device comprising:
an acquisition unit configured to scan an evaluation piece into which a specimen is processed and acquire an evaluation piece image;
a comparison result output unit configured to compare specimen information on the specimen and evaluation piece information that is obtained from the evaluation piece image and output a comparison result; and
a display unit configured to present the comparison result to a user.

20. A medical support method comprising:
by a scanner, scanning an evaluation piece into which a specimen is processed and acquiring an evaluation piece image;
by a calculation device, comparing specimen information on the specimen and evaluation piece information that is obtained from the evaluation piece image and outputting a comparison result; and
by a display device, presenting the comparison result to a user.
